(19) United States
(12) Patent Application Publication
(10) Pub. No.: US 2026/0176821 A1
(43) Pub. Date: Jun. 25, 2026

(54) A NANOCELLULOSE-BASED COMPOSITION FOR PRODUCING A FOIL OR A COATING OF A SUBSTRATE

(71) Applicant: SPACESIDE HOLDING B.V., ROTTERDAM (NL)

(72) Inventors: Lori Beth GOFF, ROTTERDAM (NL); Sander Franciscus Henricus LAMBREGTS, ROTTERDAM (NL)

(21) Appl. No.: 19/125,096

(22) PCT Filed: Nov. 1, 2023
(86) PCT No.: PCT/NL2023/050573

§ 371 Date: Apr. 28, 2025

(30) Foreign Application Priority Data
Nov. 1, 2022 (NL) 2033436

(51) Int. Cl. D21H 11/18; A61K 9/00; A61K 9/16; A61K 31/7036; A61K 31/722; C08B 3/06; C08B 37/08

(52) U.S. Cl. CPC D21H 11/18; A61K 9/0056; A61K 9/1652; A61K 31/7036; A61K 31/722; C08B 3/06; C08B 37/003; A61K 2300/00

(57) ABSTRACT

The present invention relates to a nanocellulose-based composition for producing a foil or a coating of a substrate comprising nanocellulose, a polyol and/or a polyol derivative and one or more additives. The present invention further relates to a method for preparing a nanocellulose-based intermediate product for producing a foil or a coating of a substrate. The present invention furthermore relates to a foil or coating for a substrate obtained or obtainable by the method according to the invention. The present invention finally relates to the use of the nanocellulose-based composition according to the invention, or the foil according to the invention, as packaging material, preferably a packaging material of foodstuff, packaging material of non-foodstuff, ingredient in cosmetics, and ingredient in skin care products.

### TECHNICAL FIELD

The present invention relates to the field of compostable cellulose compositions. In particular, the invention relates to a nanocellulose-based composition for producing a foil or a coating of a substrate. The described compositions comprise nanocellulose, a polyol and/or a polyol derivative, and one or more additive selected from cellulose ether chitosan, chitin, casein, zein, globulin, albumin, prolamin, glutelin, gliadin, hordein, secalin, kafirin, and avenin or combination thereof. The present invention further relates to a method for preparing the described nanocellulose-based compositions and intermediate products for producing a foil or coating of a substrate. The present invention furthermore relates to a foil or coating for a substrate obtained or obtainable by the method according to the invention. The present invention finally relates to the use of the nanocellulose-based composition according to the invention, or the foil according to the invention as packaging material, preferably a packaging material of foodstuff, packaging material of non-foodstuff, ingredient in cosmetics, and ingredient in skin care products.

### BACKGROUND

The background description includes information that may be useful in understanding the present invention. It is not an admission that any of the information provided herein is prior art or relevant to the presently claimed invention, or that any publication specifically or implicitly referenced is prior art.

Cellulose is the most abundant and widely used sustainable and renewable biopolymer on earth. The polymer chains of β-(1-4)-linked-D-glucose repeating units it consists of, are assembled into nanosized, thread-like agglomerates. These microfibrils form the structural scaffold of the fiber cell walls of plants and trees, through hydrogen bonding and van der Waals interactions. Plant/wood-based cellulose fibers have been used for centuries in materials such as papers and textiles. Various derivatives of cellulose chains have been used as binders, in food packaging, emulsifiers, and biomedical applications. During the last decades there has been an increasing interest in nanocellulose, including bacterial nanocellulose, mainly because of its abundance, high aspect ratio, mechanical properties, renewability and biocompatibility. High mechanical properties and presence of chemical reactive groups on their surfaces, allows for functionalization. Focal areas in science and technology include nanocellulose based composites, films, fibers, structural colors, barrier properties, emulsions, and viscosity tunings.

Despite the fact that nanocellulose clearly shows vast potential as a green raw material for a wide range of applications, utilization in packaging materials of food- and non-foodstuff, as a coating and as an ingredient in cosmetics and skin care products, is still limited. This is in part due to the fact that nanocellulose shows surprisingly high challenges to manage and customize the size, shape, distribution uniformity, and density of the cellulosic particles and their interactions, such that controlled, standardized, adaptable assemblies for functional materials can be manufactured. In particular, plasticity, flexibility, binding of different components, strength, water vapor, gas, and oil and grease barrier properties, and susceptibility of nanocellulose based materials to the presence of humidity or moisture are still inadequate to meet requirements for packaging materials, coating materials and as ingredient in cosmetics and skin care products.

Nanocellulose compositions have been described for example in WO 2020/201627 which relates to high consistency nanocellulose suspension for the manufacturing of films. The disclosed suspensions have a mass percentage of nanocellulose between 50-100%.

U.S. Pat. No. 10,494,493 B1 relates to the preparation of biodegradable antimicrobial packaging films by mixing nanocellulose, chitosan and glycerol, and discloses that the amount of biodegradable polymer may be from 90% to 99.9%, or from 95% to 99.9% by weight.

CN 110819175B discloses the preparation of biodegradable films from an aqueous solution obtained by mixing nanocellulose, chitosan, glycerol and TiO2, and discloses compositions with a high percentage of chitosan based on dry weight.

In light of the foregoing, new nanocellulose based compositions, products comprising such nanocellulose based compositions, and methods for producing such products, as well as uses of such products, would be highly desirable. In particular, there is a clear need in the art for sustainable, renewable nanocellulose based compositions which can be used to manufacture products that are malleable, flexible, meet requirements of strength, water, water vapor, gas, and oil and grease barrier properties, are less susceptible to the presence of humidity or moisture, while still being (home-) compostable.

Accordingly, the technical problem underlying the present invention can be seen in the provision of such nanocellulose based compositions, products comprising such nanocellulose based compositions, and methods for producing such products, as well as uses of such products, complying with any of the aforementioned needs. The technical problem is solved by the embodiments characterized in the claims and herein below.

### SUMMARY OF THE INVENTION

In a first aspect, the present invention relates to a nanocellulose-based composition for producing a foil or a coating of a substrate comprising based on dry weight:
15-40 parts nanocellulose;
15-50 parts polyol and/or a polyol derivative;
   15-45 parts one or more additive selected from a cellulose ether, chitosan, chitin, casein, zein, globulin, albumin, prolamin, glutelin, gliadin, hordein, secalin, kafirin, and avenin or combination thereof, wherein the sum of the parts adds up to one hundred parts, and wherein the composition is a dry composition, suspension, emulsion or solution, and wherein the foil or coating for a substrate is compostable, preferably home-compostable.

In a second aspect the present invention relates to a method for preparing a nanocellulose-based intermediate product for producing a foil or a coating of a substrate, the method comprising mixing nanocellulose, polyol and/or a polyol derivative and one or more additives to obtain the nanocellulose-based composition as defined in the first aspect of the invention.

In a third aspect the present invention relates to a foil or coating for a substrate obtained or obtainable by the method according to the second aspect of the invention.

In a fourth aspect the present invention relates to the use of the nanocellulose-based composition according to the first aspect of the invention, or the foil according to the third aspect of the invention as packaging material, preferably a packaging material of foodstuff, packaging material of non-foodstuff, ingredient in cosmetics, and ingredient in skin care products.

In a fifth aspect the present invention relates to the use of the nanocellulose-based composition according to the first aspect of the invention as a coating for a substrate.

### Definitions

Various terms relating to the methods, compositions, uses and other aspects of the present invention are used throughout the specification and claims. Such terms are to be given their ordinary meaning in the art to which the invention pertains, unless otherwise indicated.

Other specifically defined terms are to be construed in a manner consistent with the definition provided herein. Although any methods and materials similar or equivalent to those described herein can be used in the practice for testing of the present invention, the preferred materials and methods are described herein.

For purposes of the present invention, the following terms are defined below.

As used herein, the singular form terms “a,” “an,” and “the” include plural referents unless the content clearly dictates otherwise. Thus, for example, reference to “a bacterium” includes a combination of two or more individual bacteria, and the like.

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, etc., including the decimal numbers between the integers. As used herein, the term “at most” a particular value means that particular value or less. For example, “at most 5” is understood to be the same as “5 or less” i.e., 5, 4, 3, −10, −11, etc., including the decimal numbers between the integers.

As used herein, “comprising” or “to comprise” is construed as being inclusive and open ended, and not exclusive. Specifically, the terms and variations of the specified features, steps or components are included. These terms are not to be interpreted to exclude the presence of other features, steps or components. It also encompasses the more limiting “to consist of”.

As used herein, “as is known to the skilled person” or similar wording refers to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology, material science and engineering, such as paper and foil engineering, and related fields are well-known to those of skill in the art, and are discussed in various handbooks and literature references.

As used herein, the term ‘plasticity’ refers to the ability of the material to deform. If a material is considered to possess no or little plasticity, it is brittle, and will readily break or fall apart upon deformation.

As used herein, the term ‘flexibility’, also known as compliance, refers to the capability of a material to recover its initial form after deformation due to an applied force. If a material is considered to possess no or little flexibility, it is stiff, and will not easily deform or will resist deformation.

As used herein, the term ‘plasticizer’ refers to a substance that is added to a material to make it more flexible and/or to increase its plasticity.

As used herein, the term ‘oil and grease barrier’ refers to a barrier that prevents oil and grease from moving from one side of the barrier to the other side, through the barrier. The term ‘oil and grease’ also includes fat.

As used herein, the term ‘breaking length’ refers to a measure for the hypothetical length a foil can have at which it would break due to its own weight. The breaking length is derived from the tensile strength as measured using a tensile stretching machine and converted to a value in (kilo) meter.

As used herein, the term ‘Cobb’ refers to the water absorption by a foil or coating after a certain period of time of having (liquid) water on one side. ‘Cobb₆₀’ and ‘Cobb₁₂₀’ refer to said water absorption after respectively 60 and 120 seconds. The Cobb test is a standard method in paper making. The term “water absorption” is used herein interchangeably with the term “water uptake”.

As used herein, the term compostable refers to a material which is capable of undergoing biological decomposition in a compost site such that the material is not visually distinguishable and breaks down into carbon dioxide, water, inorganic compounds and biomass at a rate consistent with known compostable materials, and defined by the ability of conversion of a material of more than 90% of original content into minerals, water and CO₂ in less than 6 months and 90% of all particles are passable in 2×2 mm sieve (European standard of Industrial Compostability EN 13432:2000).

As used herein the term biodegradable refers to a material that is part of the earth's innate cycles like the carbon cycle and capable of decomposing back into natural elements, as defined in the European norm for Biodegradable plastics CEN/TR 159325.

As used herein, the term home-composting refers to the breaking down and composting of a material in a home compost environment, at ambient temperatures, with a natural microbial population, by humidifying the composting material. No special equipment or large volumes are required for home-compositing.

As used herein, the term industrial-composting refers to the breaking down and composting of a material in a highly controlled setting with specific temperatures, and other conditions, speeding up the process of decomposition.

As used herein, the ‘KIT’ refers to a method to determine grease/fat/oil permeability, as described in TAPPI T559. If no grease/fat/oil penetrates the sample, it obtains a KIT score of 12, which means that a mixture of heptane/toluene in a weight ratio of 1:1 doesn't penetrate the tested material.

As used herein, Moisture Vapor Transmission Rate (MVTR) refers to a measure of the passage of gaseous water through a barrier. MVTR is also known as Water Vapor Transmission Rate (WVTR), and is used here as a synonym. MVTR is measured at 23° C. and at 50% relative humidity.

As used herein, the term ‘nanocellulose’ refers to a family of nano- and micro-sized cellulosic particles, covering everything from very well defined, nanoscale cellulose nanocrystals to rather coarse fibrillated cellulose material, including microfibrillated cellulose (MFC), microcrystalline cellulose (MCC), cellulose nanocrystals (CNC), nanocrystalline cellulose (NCC), and bacterial nanocellulose (BNC).

As used herein the term ‘nanocellulose’ is understood to further include nanocellulose that has been modified, for example by changing the chemical structure of the cellulose, such as by metals and ceramics. It is understood that such modification may be performed prior to, during or after the formation of a composition, foil or coating for a substrate as defined herein.

As used herein, the term ‘foil’ refers to a thin, flexible sheet of nanocellulose based material, used as, among other things, packaging material.

As used herein, the term ‘oxygen transmission rate’ (OTR) refers to the rate at which oxygen permeates through a barrier as the result of a partial pressure difference at steady state. OTR is measured at atmospheric pressure, a relative humidity below 5%, and a temperature of 23° C.

As used herein, the term ‘porosity’ refers to the amount of air that flows through a barrier under a mild pressure difference of 1.47 kPa. Notably, this is different from the OTR, which has no pressure difference and is specific for oxygen; air permeability uses just regular air, and is measured using a Bendtsen apparatus. The skilled person will appreciate how porosity is measured using a Bendtsen apparatus.

As used herein, the term ‘Schopper-Riegler value’ (SR°), also called degree of milling, is a measure from the paper industry for the dewatering rate as defined in international standard ISO 5267.

As used herein, the term ‘stretch’ refers to how much a foil can stretch before tearing or breaking.

As used herein, the term ‘substrate’ refers to any substance or layer to which a coating, a thin layer or covering, may be applied.

### DETAILED DESCRIPTION OF THE INVENTION

The aspects of the present invention will be discussed in more detail below.

A first aspect of the present invention provides herewith a nanocellulose-based composition for producing a foil or a coating of a substrate comprising based on dry weight: 15-40 parts nanocellulose; 15-50 parts polyol and/or a polyol derivative; 15-45 parts one or more additive selected from a cellulose ether, chitosan, chitin, casein, zein, globulin, albumin, prolamin, glutelin, gliadin, hordein, secalin, kafirin, and avenin or combination thereof, wherein the sum of the parts adds up to one hundred parts, and

wherein the composition is a dry composition, suspension, emulsion or solution, and wherein the foil or coating for a substrate is compostable, preferably home-compostable. In an embodiment the one or more additives serve to: promote plasticity, increase flexibility, bind the different components in the composition, increase strength and/or improve the water, water vapor and/or gas and/or oil and grease barrier properties of the foil or the coating of the substrate.

When used herein the parts defining the composition refer to the relative amount (in weight) of the defined components of the composition based on dry weight. Thus the total parts of nanocellulose, polyol and/or polyol derivative and additive selected from a cellulose ether, chitosan, chitin, casein, zein, globulin, albumin, prolamin, glutelin, gliadin, hordein, secalin, kafirin, and avenin or combination thereof add up to one hundred parts. This means that if other components are added (e.g. a colorant, flocculation agent, metal carbonate, metal oxide, solubility aid, a substance to increase or decrease opacity as described below), these additional additives are not counted to the total of one hundred parts (based on dry weight of the composition).

The present inventors used nanocellulose as the main structural component for the nanocellulose based composition for producing a foil or a coating of a substrate. The skilled person will appreciate that any source of nanocellulose, able to form a dense network of nanocellulose particles, may be used in compositions and methods of the invention. In a preferred embodiment, the nanocellulose is selected from the group comprising microfibrillated cellulose, microcrystalline cellulose, cellulose nanocrystals, nanocrystalline cellulose, bacterial nanocellulose, plant derived nanocellulose, algae derived nanocellulose, fungi derived nanocellulose, and a combination of two or more thereof. Alternatively, any source of cellulose may be processed to form nanocellulose and be used in compositions and methods of the invention.

The inventors found that the properties of the foil or coating of a substrate produced with said nanocellulose based composition could be improved by adding polyol (and/or a polyol derivative) and certain additives. The purpose of polyol (and/or a polyol derivative) may for instance be to promote plasticity, and flexibility. When disclosed herein the total parts of polyol and/or polyol derivative is between 15-50 parts based on dry weight. Thus when a polyol and a polyol derivative are combined the total parts of the combined polyol and polyol derivative are between 15-50 parts based on the dry weight of the composition.

The inventors also found that adding specific (combinations of) binders and additives would lead to specific properties of the foil or coating of a substrate. For instance a foil consisting of just BNC and glycerol would feel oily, while a foil consisting of BNC, glycerol and a cellulose ether would not feel oily (Example 1). Further, strength and barrier properties of the foil and coating of a substrate, such as water, water vapor, gas and oil and grease barrier properties, could be improved using the proper additives.

Described herein are compositions of nanocellulose with improved properties, such as plasticity, flexibility, increased strength or increased barrier functions due to the addition of one or more additives, therefore, in an embodiment, the invention relates to composition of nanocellulose with a polyol (and/or a polyol derivative) one or more additives selected from cellulose ethers, long chain sugar polymers or proteins. The individual components are defined herein below. The invention describes a composition for producing a foil or a coating of a substrate, the composition comprising, based on the dry weight of the listed components:
15-40 parts nanocellulose, 15-50 parts of polyol and/or a polyol derivative, and 15-45 parts of one or more additive selected from a cellulose ether, chitosan, chitin, casein, zein, globulin, albumin, prolamin, glutelin, gliadin, hordein, secalin, kafirin, and avenin or combination thereof, wherein the sum of the parts adds up to one hundred parts. For example the composition may comprise 15, 16, 17, 18, 19, 20, 21, 22, 23, 24, 25, 26, 27, 28, 29, 30, 31, 32, 33, 34, 35, 36, 37, 38, 39, or 40 parts nanocellulose. For example the composition may comprise 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 parts polyol and/or a polyol derivative. For example the composition may comprise 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or 45 parts of one or more additive selected from a cellulose ether, chitosan, chitin, casein, zein, globulin, albumin, prolamin, glutelin, gliadin, hordein, secalin, kafirin, and avenin or combination thereof.

Further disclosed herein are compositions comprising:
15-70 parts, preferably 15-65 parts, more preferably 15-60 parts nanocellulose even more preferably 15-55 parts, such as between 15-50, or between 15-45 or between 15-40 parts cellulose ether; and
15-50 parts, preferably 15-45 parts, more preferably 15-40 parts polyol and/or a polyol derivative; and
10-60 parts additive selected from a cellulose ether, chitosan, chitin, casein, zein, globulin, albumin, prolamin, glutelin, gliadin, hordein, secalin, kafirin, and avenin or combination thereof;
wherein the composition is a dry composition, suspension, emulsion or solution, and wherein the foil or coating for a substrate is compostable, preferably home-compostable.

### Improved Plasticity and Flexibility

The inventors found in particular that adding a polyol and/or polyol derivative such as glycerol would promote plasticity and flexibility of a foil or coating of a substrate. In addition, if no other additives were added, the surface of the foil or coating of a substrate would have an oily, sticky feel (Example 1). Oiliness or stickiness of the foil or coating of a substrate resulted in an increased adherence to other materials. Another polyol, mannitol, was also qualitatively evaluated. The inventors found that mannitol would yield a similar effect on plasticity and flexibility as glycerol, but would feel less oily, which is preferable in case of use of the foil for, for instance, a sandwich bag. The inventors further found that mannitol can replace chitosan (see below under Increased water vapor and water barrier properties) in compositions with BNC, MHEC, HEC and glycerol while the WVTR is maintained optimal, albeit that the water stability of a foil made from such compositions would be poorer than a foil from compositions including chitosan. After being submerged in water for an hour, a foil from compositions including mannitol instead of chitosan could be taken apart with no effort. This would be expected since mannitol is easily water soluble. The skilled person will appreciate that other polyols such as propylene glycol and a combination of such polyols would have similar effects. Therefore in an embodiment polyol and/or polyol derivative is added to improve plasticity and flexibility. In an embodiment the polyol is an organic compound containing multiple hydroxyl groups. Preferably the polyol is a low molecular weight polyol or a sugar alcohol. Non-limiting examples of low molecular weight polyols are glycerol, trimethylolpropane, pentaerythritol, polypropylene glycol, or a low molecular weight polyol-diol such as 1,4-butanediol. When used herein sugar alcohols are a class of low molecular weight polyols which are commonly obtained by hydrogenation of sugars. Non-limiting examples are compounds with the formula (CHOH)ₙH₂, where n=4-6. Non-limiting examples of sugar alcohols are maltitol, mannitol, sorbitol, xylitol, erythritol, and isomalt. In an embodiment the polyol or polyol derivative is not a polymer.

A polyol derivative, triacetin (glycerol triacetate), was qualitatively evaluated. The inventors found that triacetin would yield a similar effect on plasticity and flexibility as glycerol. The inventors also found that a mixture of triacetin and glycerol would yield a similar effect on plasticity and flexibility as glycerol (see Example 10). Therefore, in an embodiment the polyol derivative is a polyol as defined herein wherein one or more of the —OH groups have been replaced by a functional group. For example the —OH group may be replaced by an ester or an ether. Thus in an embodiment the polyol derivative is a polyol as defined herein wherein one or more —OH groups are replaced by an ether of a linear or branched C₁₋₈ alkanol or alkenol or replaced by an ester of a linear or branched C₁₋₈ alkanoic acid or alkenoic acid. A non-limiting and preferred example of a polyol derivative is triacetin (glycerol triacetate).

Preferred options are a polyol selected from the group consisting of propylene glycol, glycerol, mannitol, or wherein the polyol derivative is triacetin or a combination of two or more thereof.

The inventors found that mannitol could be used as a polyol, promoting plasticity and flexibility as would glycerol, and that also a combination of mannitol and glycerol would have such an effect.

### Increased Strength

The inventors further found that additives such as cellulose ethers such as HEC (hydroxyethyl cellulose) and MHEC (methyl hydroxyethyl cellulose) or other additives such as cellulose ether, chitosan, chitin, casein, zein, globulin, albumin, prolamin, glutelin, gliadin, hordein, secalin, kafirin, and avenin or combination thereof provide significantly improved breaking length and directly proportional tensile strength, as well as an improved look and feel of the foil and coating of a substrate. The inventors worked with viscosities of 10000-70000 mPa·s (2% in water at room temperature), but the skilled person will appreciate that higher and lower viscosities would yield similar results. The skilled person will further appreciate that other cellulose ethers may be used. Thus in an embodiment the cellulose ether is ionic cellulose ether or non-ionic cellulose ether, in particular carboxymethyl cellulose (CMC), ethyl hydroxyethyl cellulose, and methyl ethyl hydroxyethyl cellulose hydroxyethyl cellulose (HEC), and methyl hydroxyethyl cellulose (MHEC) or a combination thereof. Therefore in an embodiment an additive is added to improve tensile strength, wherein the additive is a cellulose ether, preferably wherein the cellulose ether is selected from the group comprising ionic cellulose ether or non-ionic cellulose ether, or a combination thereof, more preferably selected from the group consisting of carboxymethyl cellulose (CMC), hydroxyethyl cellulose (HEC), methyl hydroxyethyl cellulose (MHEC), ethyl hydroxyethyl cellulose, methyl ethyl hydroxyethyl cellulose, hydroxypropyl methylcellulose, or a combination of two or more thereof.

The inventors also found that with no additional measures foils could be made with a thickness of between 10-150 micron. The skilled person will appreciate that, with the right equipment, other thicknesses can readily be achieved.

The inventors found furthermore that using chitosan as part of the additive or as the entire additive component would produce mechanically tougher foils and coatings of a substrate, as measured by their tensile strength, compared to foils and coatings of a substrate made out of compositions comprising MHEC and/or HEC and/or glycerol only (Examples 2 and 3). It is understood that chitosan can partly or completely replace the cellulose ether as an additive. The inventors used mushroom derived chitosan (as it is vegan and guaranteed free of allergens), but the skilled person will appreciate that any source of chitin (from which chitosan is derived), chitosan and chitosan derivatives may be used, such as from marine animals and chemically synthesized types of chitosan. The skilled person will further appreciate that milk or plant proteins such as casein, zein, globulin, albumin, prolamin, glutelin, gliadin, hordein, secalin, kafirin, and avenin can be used instead of or in addition to chitosan and/or a cellulose ether. Therefore in an embodiment an additive is added to improve tensile strength, wherein the one or more additive is selected from chitosan, chitin, casein, zein, globulin, albumin, prolamin, glutelin, gliadin, hordein, secalin, kafirin, or avenin or a combination thereof. Preferably the chitosan is selected from vegetal chitosan, fungal chitosan, synthesized chitosan, chitosan made water soluble or a combination of two or more thereof. The inventors utilized acidic water to dissolve chitosan. The skilled person will appreciate that other acids can be used for this purpose. The inventors worked with chitosan with viscosities between 20 and 200 cps (mPa·s) (1% in 1% acetic acid at room temperature), but the skilled person will appreciate that other viscosities, in particular higher viscosities, will also work. The skilled person is also aware that chitosan has conservation, in particular antifungal, properties.

It is further envisioned that particularly preferred may be combinations of chitosan with an additional additive, as the combination of chitosan with an additional additive may further improve the mechanical properties of a foil made from the described compositions. Thus in an embodiment, the invention describes a nanocellulose-based composition for producing a foil or a coating of a substrate comprising nanocellulose, a polyol and/or polyol derivative, and one or more additives, wherein the composition comprises based on dry weight: 15-40 parts nanocellulose; 15-50 parts polyol or a polyol derivative or a combination thereof; 15-45 parts one or more additive
wherein the additive comprises 1-44, preferably 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 parts of chitosan and 1-44 parts, preferably 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, or 44 parts of an additional additive selected from a cellulose ether, chitin, casein, zein, globulin, albumin, prolamin, glutelin, gliadin, hordein, secalin, kafirin, and avenin or combination thereof, together adding up to 15-45 parts; wherein the composition is a dry composition, suspension, emulsion or solution, and wherein the foil or coating for a substrate is compostable.

### Increased Water Vapor and Water Barrier Properties

The present inventors found that nanocellulose-based compositions comprising cellulose ethers such as MHEC and/or chitosan as an additive, in addition to plasticizers such as a polyol and/or polyol derivative, would also result in foils and compositions with improved water vapor barrier properties (Example 4).

The skilled person will appreciate that other cellulose ethers, such as ionic cellulose ether or non-ionic cellulose ether, in particular carboxymethyl cellulose (CMC), ethyl hydroxyethyl cellulose, and methyl ethyl hydroxyethyl cellulose or a combination of two or more ethers would have similar effects as hydroxyethyl cellulose (HEC), and methyl hydroxyethyl cellulose (MHEC). Therefore in an embodiment an additive is added to improve the water vapor barrier properties, wherein the additive is a cellulose ether, preferably wherein the cellulose ether is selected from the group comprising ionic cellulose ether or non-ionic cellulose ether, or a combination thereof, more preferably selected from the group consisting of carboxymethyl cellulose (CMC), hydroxyethyl cellulose (HEC), methyl hydroxyethyl cellulose (MHEC), ethyl hydroxyethyl cellulose, methyl ethyl hydroxyethyl cellulose, hydroxypropyl methylcellulose, or a combination of two or more thereof.

Further foils and coatings of a substrate manufactured from BNC, MHEC and/or HEC, and chitosan, with or without a polyol and/or polyol derivative such as glycerol could be submerged for at least a month in water (room temperature) and would remain intact. Therefore it is concluded that the inclusion of chitosan as at least one of the additives within the above specified ranges renders a foil or coating water resistant even in the absence of a polyol or polyol derivative.

The inventors found that foils and coatings of a substrate made with a nanocellulose based-composition comprising MHEC, polyol (and/or a polyol derivative) and chitosan, would be even more water repellent, as testified by Cobb values≤19 g/m², compared to where no chitosan was used (Example 6). The skilled person will appreciate that this effect will also be achieved if a different cellulose ether and/or no or a different plasticizer is used.

Including chitosan in compositions would also improve water vapor and water barrier properties of foils and coatings of a substrate made out of such compositions, compared to foils and coatings of a substrate made out of compositions comprising MHEC and/or HEC and glycerol only (Example 5). Foils and coatings of a substrate comprising Chitosan hydrochloride (also called Chitosan HCl or chitosan made water soluble, by modifying the chitosan by exposing it to, for example HCl, it is rendered soluble in water) showed better water vapor and water barrier properties compared to foils and coatings of a substrate made without chitosan or chitosan HCl (Examples 2, 3 and 7). The inventors further tested the water stability of a foil made of a composition comprising Chitosan HCl (HEC, glycerol, and BNC, in (agitated) water at room temperature. The foil retained its strength after a month of soaking. The inventors used mushroom derived chitosan (as it is vegan and guaranteed free of known allergens), but the skilled person will appreciate that any source of chitosan and chitosan derivatives may be used, such as from marine animals and chemically synthesized types of chitosan. The skilled person will further appreciate that proteins such as casein, zein, globulin, albumin, prolamin, glutelin, gliadin, hordein, secalin, kafirin, and avenin can be used instead of or in addition to chitosan. Therefore when used herein the term long chain sugar polymer or protein should be interpreted to refer to long chain sugar polymers such as chitosan, chitin or similar products but to exclude nanocellulose or cellulose ethers, the term further encompasses proteins such as casein, zein, globulin, albumin, prolamin, glutelin, gliadin, hordein, secalin, kafirin, and avenin or similar proteins. Herein similar products or proteins are intended to refer to long chain sugar polymers or proteins that confer the same or similar properties to the composition as described herein.

Foils and coatings of a substrate manufactured from compositions which include chitosan could be stored 24+h submerged in water (room temperature) and would remain intact, retaining their strength, although some of the glycerol would dissolve in the water, which would make the foils slightly less flexible. Further, the inventors found that a bowl formed from a foil of chitosan composition could hold boiling water, which would be at room temperature after 30 minutes, for over a week, and an acidic, carbonated liquid for over 72 hours. (Example 8).

Therefore in an embodiment an additive is added to improve water vapor and water barrier properties, wherein the one or more additive is selected from chitosan, chitin, casein, zein, globulin, albumin, prolamin, glutelin, gliadin, hordein, secalin, kafirin, and avenin or a combination thereof, preferably wherein the chitosan is selected from vegetal chitosan, fungal chitosan, synthesized chitosan, chitosan made water soluble or a combination of two or more thereof.

Using mannitol instead of glycerol would result in an improved MVTR, compared to nanocelluloses based composition wherein no chitosan was used, although this would also result in a less water repellent foil or coating of a substrate (Example 4).

### Improved Gas Barrier Properties

The inventors found that adding chitosan HCl (hydrochloride) or similar compounds such as but not limited to chitosan, chitin, casein, zein, globulin, albumin, prolamin, glutelin, gliadin, hordein, secalin, kafirin, and avenin or combination thereof, to the nanocellulose based composition would substantially improve gas barrier properties (Example 7) of foils and coatings of a substrate made from the composition in addition to maintaining good water vapor barrier properties (Example 4 and 6). The oxygen transmission barrier (OTR) was markedly lower compared to the OTR of foils and coatings of a substrate made from compositions where regular chitosan was used (Example 7). The skilled person will appreciate that the modification of regular chitosan to arrive at the chitosan HCl derivative can be performed in many ways (Minh et al. 2019).

The skilled person will also understand that other modifications to make chitosan soluble will work similarly.

Therefore in an embodiment an additive is added to improve gas barrier properties, wherein the one or more additive is chitosan made water soluble or a combination of chitosan made water soluble and one or more additive selected from chitosan, chitin, casein, zein, globulin, albumin, prolamin, glutelin, gliadin, hordein, secalin, kafirin, and avenin. Preferably the chitosan is selected from vegetal chitosan, fungal chitosan, synthesized chitosan, or a combination of two or more thereof.

### Improved Water Vapor Barrier and Gas Barrier Combined

The inventors found that foils and coatings of a substrate made with a nanocellulose-based composition comprising HEC, polyol and/or a polyol derivative and chitosan had improved water vapor barrier and gas barrier properties combined (Example 3).

Therefore, in an alternative embodiment the invention relates to a nanocellulose-based composition for producing a foil or a coating of a substrate comprising nanocellulose, a polyol and/or polyol derivative; and one or more additives, wherein the one or more additives are selected from a cellulose ether, chitosan, chitin, casein, zein, globulin, albumin, prolamin, glutelin, gliadin, hordein, secalin, kafirin, or avenin or combination thereof,
preferably wherein the polyol and/or polyol derivative is selected from the group comprising propylene glycol, glycerol, mannitol, triacetin and a combination of two or more thereof; and/or
preferably wherein the cellulose ether is an ionic cellulose ether or a non-ionic cellulose ether, or a combination thereof, more preferably wherein the cellulose ether is selected from the group comprising carboxymethyl cellulose (CMC), hydroxyethyl cellulose (HEC), methyl hydroxyethyl cellulose (MHEC), ethyl hydroxyethyl cellulose, methyl ethyl hydroxyethyl cellulose, hydroxypropyl methylcellulose, or a combination of two or more thereof; and/or
preferably wherein the chitosan is selected from vegetal chitosan, fungal chitosan, synthesized chitosan, chitosan made water soluble or a combination of two or more thereof,
wherein the composition is a dry composition, suspension, emulsion or solution, and wherein the foil or coating for a substrate is compostable, preferably home-compostable.

### Compostability

The present inventors found further that adding the above described additives, didn't impede, but maintained the ability of the foil or coating for a substrate to be transformed into compost through a composting process.

The present inventors found that foils made from any of the various compositions were mostly degraded after two weeks and fully degraded after six weeks, in a home composting setting. Industrial composting would likely result in even faster degradation.

Additionally the present inventors found that all foils made from any of the various compositions were semi-transparent. It was possible to look through the foils and read text through the foils, but only if the text was placed directly underneath the foils. The foils look like “frosted glass”. All the foils were colourless to white or light yellow.

The inventors found further that colorants could successfully be added to the foil or coating of a substrate. Flocculation agents like fumed silica created a more cloudy look, and calcium carbonate/calcium oxide produced a more white sheet, while retaining a good OTR and a KIT value of 12.

Therefore in an embodiment an additional additive is added selected from the group comprising colorant, flocculation agent, metal carbonate, metal oxide, solubility aid, a substance to increase or decrease opacity, and a combination of two or more thereof.

The skilled person will appreciate that the nanocellulose based composition may be a dry composition or may be a suspension or solution in liquid.

Therefore in an embodiment the nanocellulose based composition is a suspension or solution in a liquid, wherein the liquid comprises water, an alcohol, or a mixture thereof.

The inventors further found that prolonged exposure to temperatures over 120° C. would result in discoloration and eventually brittleness of foils and coatings of a substrate. Temperatures up to 95° C. had no detrimental effects (both as drying temperature as well as when dry), regardless of whether temperatures were obtained by convection heating or radiation heating.

The inventors also found that all foils could be sealed. Sealing properties were invoked by applying a droplet of water, ethanol, propylene glycol (and likely many other polar liquids) or a mixture of water and, for instance, HEC or MHEC, on (part of) a foil, pressing another (part of) a foil to the moistened foil, and subsequently heating the foils to evaporate the liquid or leaving the foils to dry under ambient conditions. Additionally the inventors found that the foil or coating for a substrate may be sealed using adhesives common in the packaging industry.

In addition it was found that it was possible to print the foils using an ink-jet printer for home use and through screen printing techniques.

The inventors further found that certain combinations of additives resulted in a foil or coating of a substrate in which both the water or water vapor barrier and the oxygen barrier were improved, while maintaining a grease/fat/oil and grease barrier of the highest degree and still being compostable.

The water vapor barrier and the oxygen barrier of coatings of a substrate made with a nanocellulose-based composition comprising a polyol and/or a polyol derivative and the additives cellulose ether and chitosan were both markedly improved compared to foils and coatings of a substrate made from other combinations of additives.

Therefore, in a preferred embodiment, the composition comprises a polyol and/or a polyol derivative and one or more additive selected from HEC, MHEC and chitosan, more preferably wherein the composition comprises based on dry weight: 10-50, preferably 15-40, such as 15, 16, 17, 17, 18, 19, 20, 21, 22, 23, 24, 25, 26, 27, 28, 29, 30, 31, 32, 33, 34, 35, 36, 37, 38, 39 or 40 parts nanocellulose, 10-60, preferably 15-50, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 parts polyol and/or a polyol derivative, preferably glycerol, 10-55, preferably 15-45, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45 parts of one or more additive, including the decimal numbers between the integers. In a preferred embodiment the one or more additive is selected from HEC, MHEC and chitosan or a combination thereof.

Preferably when used herein parts, when referring to parts of a specific component based on dry weight, refers to a dry weight percentage of the component, meaning that the sum of parts of the components adds up to one hundred parts. It is herein understood that the percentage of weight (or the parts as used herein) is defined based on the amount of nanocellulose, polyol and/or polyol derivative and additive. Therefore when using weight or parts ranges not included are components added to for example add color, increase or decrease transparency, etc.

A second aspect of the present invention provides herewith a method for preparing a nanocellulose-based intermediate product for producing a foil or a coating of a substrate, comprising the nanocellulose composition according to the first aspect of the invention, the method comprising mixing nanocellulose, polyol (and/or a polyol derivative) and one or more additive to obtain the nanocellulose-based composition according to the invention. In an embodiment the one or more additive serves to: promote plasticity, increase flexibility, bind the different components in the composition, increase strength and/or, improve the water, water vapor and/or gas and/or oil and grease barrier properties of the foil or the coating of the substrate.

### Foil Preparation Methods

In order to make the foils and coatings for a substrate, the inventor mixed nanocellulose, which may have been purified and pulverized, with the additives.

Therefore, in a preferred embodiment, the nanocellulose is pulverized prior to, during or after blending with the one or more additive.

The inventors found further that for making a foil or coating for a substrate from the nanocellulose-based composition a nanocellulose-based suspension or solution had to be obtained from the composition.

Therefore, in a preferred embodiment, the dry nanocellulose-based composition is suspended or dissolved in a liquid to obtain a nanocellulose-based suspension or solution.

The inventors found that the nanocellulose-based suspension or solution could then be applied to a selected surface, and subsequently the liquid could then be removed.

The skilled person will appreciate that the removal of the liquid can be done using a number of different techniques, possibly combined, such as simply pressing, drying in an oven or microwave, vacuum water suction, and steam heated drums.

Therefore, the method may comprise the additional steps of: applying the nanocellulose-based suspension or solution to a surface or a substrate, and removing partly or completely the liquid from the nanocellulose-based suspension or solution, to obtain a nanocellulose-based foil or coating for a substrate.

The inventors also found that the nanocellulose was preferably kept in an acidic or basic aqueous suspension, in the range of 4.5-11 pH, in order to prevent that fine fiber networks would be formed permanently. In particular, from nanocellulose kept in a suspension of pH 4.5, 7, 10 and 11 a stable foil could be produced, both directly after preparing the suspension or after leaving the nanocellulose in the suspension for 7 days. At day 7 all suspensions were free from visible bacterial growth. At day 12 the pH 4.5 and 7 suspension showed bacterial growth, the pH 10 and 11 were still free from visible bacterial growth.

In an embodiment, the pH of the solution or suspension is adjusted to 5 to 8 pH by exposing the slurry to a neutralization agent.

A third aspect of the present invention provides herewith a foil or coating for a substrate obtained or obtainable by the method according to the invention.

The inventors found that foils and coatings for a substrate made with the nanocellulose based composition of the present invention resulted in foils and coatings for a substrate with properties which allowed the foils and coatings for a substrate to be used as packaging material of foodstuff, and packaging material of non-foodstuff. Specifically, foils and coatings for the substrate could be made, having an air permeability below 10 mL/min, preferably below 1 mL/min, as measured by the Bendtsen method using a pressure difference of 1.5 kPa; and/or an oxygen transmission rate below 10 mL/(m²*day), preferably below 1 mL/(m²*day), as measured at 23° C., <5% RH of the carrier and testing gas, atmospheric pressure, and/or a grease/fat/oil permeability (KIT) score of at least 10, preferably 12.

Therefore, in a preferred embodiment, the foil or coating for the substrate has
air permeability below 10 mL/min, preferably below 1 mL/min, as measured by the Bendtsen method using a pressure difference of 1.5 kPa; and/or
an oxygen transmission rate below 10 mL/(m²*day), preferably below 1 mL/(m²*day), as measured at 23° C., <5% RH, atmospheric pressure, and/or
a grease/fat/oil permeability (KIT) score of at least 10, preferably at least 12.

The inventors further found that foils and coatings for a substrate made with the nanocellulose based composition of the present invention could be readily impregnated.

The skilled person will appreciate that for impregnation any impregnation substance may be used, such as but not limited to PBS, PHA, PHB, calendula, oil-based, wax-based, ceramics-based, and metallic-based impregnation substances. The inventors found that in addition to barrier properties impregnation substances also affect other properties such as transparency and printability.

Therefore, in a preferred embodiment, the foil or coating for a substrate is impregnated with a substance.

The inventors found further that foils and coatings for a substrate made with the nanocellulose based composition of the present invention could be readily coated. The inventors found that foils coated with wax became maximal water repellent by the wax treatment, leading to a Cobb₆₀-value of 0 g/m². (Example 6).

The skilled person will appreciate that for coating any coating substance, preferably a compostable coating substance, may be used, such as oil-based and wax-based coating substances, however non-compostable coatings may also be used.

Therefore, in a preferred embodiment, the foil is coated with a coating.

A fourth aspect of the present invention provides herewith the use of the nanocellulose-based composition according to the invention, or the foil according to the invention as packaging material, preferably a packaging material of foodstuff, packaging material of non-foodstuff, ingredient in cosmetics, and, ingredient in skin care products.

The inventors found that with a low air permeability, a low oxygen transmission rate, and a low grease/fat/oil permeability the foil of the present invention, made with the nanocellulose-based composition of the present invention was ideally suited to be used as packaging material, both for foodstuff and for non-foodstuff, such as but not limited to cosmetic packaging, packaging for sporting accessories, clothing, and packaging for medical products.

The inventors further found that the nanocellulose-based composition, due to the aforementioned properties, was also ideally suited to be used as an ingredient in cosmetics and in skin care products.

The inventors finally found that the nanocellulose based composition could itself be used as a coating for a substrate.

Therefore, in a preferred embodiment, the use of the nanocellulose-based composition according to the invention is as a coating for a substrate.

The invention is further detailed by the accompanying examples, which are exemplary and explanatory of nature and are not limiting the scope of protection given to the invention. To the person skilled in the art it may be clear that many variants may be conceivable falling within the scope of protection, defined by the present claims.

### Examples

### Main Findings:

The below described experiments resulted in the following main findings:

### Plasticity

Adding glycerol promotes plasticity of a foil or coating of a substrate.
Glycerol can be partly or completely replaced by mannitol or triacetin to promote plasticity of a foil or coating of a substrate.

### Increased Strength

Addition of HEC or MHEC provides significantly improved tensile strength (Example 2).
HEC and/or MHEC, glycerol and chitosan provide significantly improved tensile strength compared to MHEC and/or HEC and glycerol but no chitosan (Example 2 and 3).
HEC and/or MHEC, glycerol and chitosan HCl (hydrochloride), also results in improved tensile strength, especially compared to foils and coatings of a substrate made without chitosan or chitosan HCl, although less than when regular chitosan is used (Example 2 and 3).

### Water Vapor Barrier

MHEC and HEC combined improves water vapor barrier properties if mannitol and glycerol are added, or only MHEC and HEC combined is added with no other additives (Example 4).
MHEC or HEC, glycerol and chitosan improves water vapor barrier properties (Example 4).
Chitosan and glycerol improves water vapor barrier properties (Example 4).

### Water Resistance

MHEC and HEC combined compositions could be submerged in water for <1 hour (no stirring), without disintegrating.
MHEC and/or HEC, glycerol and chitosan were significantly more resistant to water penetration and weakening due to water than when no chitosan was used. (Example 5)

### Oxygen Barrier

Chitosan HCl (hydrochloride) substantially improves oxygen barrier properties, in addition to maintaining good water vapor barrier properties (Example 7).
MHEC and/or HEC and/or chitosan, with or without glycerol improves oxygen barrier properties (Example 7)

### Combination of Water Vapor Barrier and Gas Barrier

Combination of the additives HEC, glycerol and chitosan results in improved water vapor barrier and gas barrier properties combined (Example 3).

### Methods and Materials:

All samples were 4 mm thick initially (i.e. wet thickness), prepared on a silicon sheet (unless indicated otherwise) and allowed to dry.
HEC=hydroxyethyl cellulose (powder)

$$MHEC = methyl hydroxyethyl cellulose ( powder )$$

$$Chitosan = regular mushroom derived chitosan ( powder )$$

$$Chitosan water soluble = regular mushroom derived chitosan post - treated with HCl ( powder ) , also called Chitosan HCl$$

The following recipes were tested:

TABLE 1
nanocellulose-based compositions tested - indicated are ranges of percentages within
compositions are tested for each recipe in the experiments below (percentages based
on dry weight, total of listed ingredients for each recipe adds up to 100%).

| Recipe | | | | | | Chitosan | |
|---|---|---|---|---|---|---|---|
| no. | BNC | MHEC | HEC | Glycerol | Chitosan | HCl | Mannitol |
| 1 | 20-70% | 10-50% | 10-50% | | | | |
| 2 | 15-40% | 10-30% | 10-30% | 20-50% | | | |
| 3 | 15-40% | 10-30% | 10-30% | 15-35% | | | 15-35% |
| 4 | 15-40% | | 10-40% | 20-50% | 10-40% | | |
| 5 | 15-40% | 10-40% | | 20-50% | 10-40% | | |
| 6 | 15-40% | | | 20-50% | 20-45% | | |
| 7 | 15-40% | 20-45% | | 20-50% | | | |
| 8 | 15-40% | | 20-45% | 20-50% | | | |
| 9 | 15-40% | | 10-40% | 20-50% | | 10-40% | |
| 10 | 30-50% | | | 50-70% | | | |
| 11 | 50-70% | | | | 30-50% | | |

Exemplary compositions falling within the recipe ranges are provided below:

| Recipe | BNC | MHEC | HEC | Glycerol | Chitosan | Chitosan | Mannitol |
|---|---|---|---|---|---|---|---|
| no. | (%) | (%) | (%) | (%) | (%) | HCl (%) | (%) |
| 1 | 48 | 30 | 22 | | | | |
| 2 | 30 | 16 | 10 | 44 | | | |
| 3 | 35 | 15 | 15 | 20 | | | 15 |
| 4 | 26 | | 14 | 29 | 31 | | |
| 5 | 29 | 27 | | 27 | 17 | | |
| 6 | 30 | | | 35 | 35 | | |
| 7 | 23 | 27 | | 50 | | | |
| 8 | 26 | | 24 | 50 | | | |
| 9 | 29 | | 14 | 38 | | 19 | |
| 10 | 39 | | | 61 | | | |
| 11 | 70 | | | | 30 | | |

### Oily/Sticky

### Example 1

| Foil | Feels |
|---|---|
| Recipes 1, 2, 3, 7 and 8 | Not oily/not sticky |
| Recipe 10 | Oily/sticky |

General Feeling when Moving a Finger Over the Foil

### Increased Strength

### Example 2: Tensile Strength Tests

| Tests recipe no.: | Tensile strength (kN/m) |
|---|---|
| Recipe 7 | 1.23 |
| Recipe 7 (prepared on PTFE) | 1.51 |
| Recipe 8 | 0.66 |
| Recipe 6 | 2.04 |
| Recipe 5 | 2.12 |
| Recipe 5 (prepared on PTFE) | 1.93 |
| Recipe 9 | 1.65 |
| Recipe 2 | 0.70 |
| Recipe 2 (6 mm instead of 4 mm | 0.61 |
| thickness) | |
| Recipe 2 (6 mm instead of 4 mm | 0.87 |
| thickness) | |

### Example 3

| Recipe No.: | Tensile strength | | WVTR | | OTR | |
|---|---|---|---|---|---|---|
| Recipe 5 | 2.1 | kN/m | 231 | g/m²/day | | |
| Recipe 9 | 1.65 | kN/m | 484 | g/m²/day | 0.6 | mL/m²/day |
| Recipe 3 | | | 251 | g/m²/day | 27 | mL/m²/day |
| Recipe 2 | 0.7 | kN/m | 411 | g/m²/day | 4.2 | mL/m²/day |

| | | | | |
|---|---|---|---|---|
| Recipe 4 | 271 | g/m²/day | 0.4 | mL/m²/day |

### Water Vapor Barrier

### Example 4: Water Vapor Transmission Rate Tests

| Tests (recipes) | WVTR (g/m²/day) |
|---|---|
| Recipe 7 | 433.8 |
| Recipe 7 (prepared on PTFE) | 473.6 |
| Recipe 8 | 463.2 |
| Recipe 4 | 270.6 |
| Recipe 6 | 270.8 |
| Recipe 5 | 231.0 |
| Recipe 5 (prepared on PTFE) | 202.0 |
| Recipe 9 | 483.6 |
| Recipe 2 | 410.6 |
| Recipe 2 (6 mm instead of 4 mm thickness) | 449.2 |
| Recipe 2 (6 mm instead of 4 mm thickness) | 436.2 |
| Recipe 1 | 272.2 |
| Recipe 3 | 250.6 |

### Water Resistance

### Example 5: Water Uptake Properties of Foils Made of a Nanocellulose-Based Composition

In addition to a foil made of a nanocellulose based composition consisting of BNC, MHEC, HEC, glycerol, a foil made of a nanocellulose based composition consisting of BNC, MHEC, glycerol and chitosan, was tested. Parameter tested was Cobb₁₂₀. Cobb₁₂₀ was measured after 120 seconds of exposure to water.

| | Cobb₁₂₀ |
|---|---|
| Foil | [g/m²] |
| Recipe 2 | 157 |
| Recipe 5 | 19 |

The foil made of a nanocellulose based composition consisting of BNC, MHEC, glycerol, and chitosan resulted in a significantly lower Cobb value, 19 (after 120 seconds exposure) compared to the foil without chitosan; 82±12 (after 60 seconds exposure).

### Example 6: Barrier Properties of Coated Foils Made of a Nanocellulose-Based Composition

The foils were coated for these experiments with a coating from Alkyl Ketene Dimer wax (AKD wax). The foils were coated with a thin or a thick coating. Coated foils also became maximal water repellent by the wax treatment, and had a determined Cobb₆₀-value of 0 g/m².

### Gas Barrier

### Example 7: Oxygen Transmission Rate Tests

| Tests (recipes) | OTR (cm³/(m²*day)) |
|---|---|
| Recipe 7 | 11.8 |
| Recipe 7 (prepared on PTFE) | 10.6 |
| Recipe 8 | 5.2 |
| Recipe 4 | 0.4 |
| Recipe 9 | 0.6 |
| Recipe 2 | 4.2 |
| Recipe 2 (6 mm instead of 4 mm thickness) | 28.2 |
| Recipe 2 (6 mm instead of 4 mm thickness) | 3.4 |
| Recipe 1 | 3.2 |
| Recipe 3 | 26.6 |

### Example 8: Cups Made from BNC Material

A cup was formed using a material made according to any of recipes no. 4, 5 or 11 and tested for its ability to hold different beverages.

The cup was filled with boiling water, which was allowed to cool down over the course of 30 minutes to room temperature. The cup was left with the liquid for a week and inspected. No leakage was observed during the observation period; the cup remained intact.

Next a similar cup was filled with an acidic carbonated liquid and left for 72 hours. No leakage was observed during the observation period; the cup remained intact.

### Example 9—Chitosan Comprising Compositions

The following recipes were prepared (percentages based on dry weight, the sum of all percentages adds up to 100%):

| | | | Chitosan HCl | Chitosan | |
|---|---|---|---|---|---|
| | BNC | glycerin | (water sol.) | (acid sol.) | MHEC |
| # | (%) | (%) | % | % | (%) |
| 150 | 30-40 | 30-40 | — | 5-10 | 20-30 |
| 151 | 50-70 | 15-30 | — | 1-14 | 1-20 |
| 152 | 41-50 | 2-14 | 36-45 | — | — |
| 153 | 25-40 | 15-30 | 36-45 | — | — |
| 154 | 15-25 | 30-40 | 36-45 | — | — |
| 155 | 5-14 | 40-50 | 36-45 | — | — |
| 156 | 30-40 | 40-50 | 15-20 | — | — |
| 157 | 30-40 | 25-40 | 20-30 | — | — |
| 158 | 30-40 | 15-30 | 30-45 | — | — |
| 159 | 30-40 | 2-14 | 46-68 | — | — |

Samples 151, 152, 155, and 159 are reference examples.

Exemplary compositions falling within the recipe ranges are provided below:

| | | | Chitosan HCl | Chitosan | |
|---|---|---|---|---|---|
| Example | BNC | glycerin | (water sol.) | (acid sol.) | MHEC |
| no. # | (%) | (%) | % | % | (%) |
| E150 | 32 | 37 | — | 7 | 24 |
| E151 | 64 | 20 | — | 7 | 9 |
| E152 | 48 | 10 | 42 | — | — |
| E153 | 32 | 25 | 43 | — | — |
| E154 | 16 | 40 | 44 | — | — |
| E155 | 8 | 49 | 43 | — | — |
| E156 | 36 | 48 | 16 | — | — |
| E157 | 36 | 34 | 30 | — | — |
| E158 | 36 | 24 | 40 | — | — |
| E159 | 36 | 8 | 56 | — | — |

The different preparations were tested qualitatively and tested for strength, elasticity, Oxygen Transmission Rate and Water Vapor Transmission Rate.

### Results:

### Observations

Sheet 151 was very “crinkly”; the appearance looked like the fibers were clogged together, unlike all other sheets with lower BNC. Sheet 159 was very stiff/“crinkly” and very yellow. (In the series 156-159, the ‘yellowness’ increases with chitosan concentration).

### Soaking in Water for 1 h (Room Temperature)

Sample 152, 155 and 159 were not stable; they disintegrated when removing from the water, just by lifting it. The other samples were stable and could resist (gentle) pulling. (Sample 151 failed the gentle pulling test after 1.5 week in water; the others were stable when the test was aborted after 1.5 week). Summarizing: samples 152, 155 and 159 have undesired properties that give them poor resistance to water.

The remaining tests are summarized in the Table below

| | Force at | Elongation | OTR | WVTR |
|---|---|---|---|---|
| # | break (N) | at break (%) | (mL/m2/day) | (g/m2/day) |
| 150 | 23.4 | 4.7 | 2.7 | 172.2 |
| 151 | 19.3 | 1.5 | 5574.9 | 102.0 |
| 152 | 23.5 | 4.6 | <1 | 201.8 |
| 153 | 22.8 | 5.3 | <1 | 240.0 |
| 154 | 18.7 | 4.4 | <1 | 313.2 |
| 155 | 2.0 | 0.7 | <1 | 336.4 |
| 156 | 16.8 | 5.8 | 1.3 | 407.6 |
| 157 | 22.5 | 2.2 | <1 | 332.0 |
| 158 | 20.8 | 5.3 | <1 | 293.4 |
| 159 | 26.5 | 3.1 | <1 | 289.4 |

Most notably, reference sample 151 has a poor oxygen barrier, and reference sample 155 has poor tensile properties.

### Example 10

Two compositions were made comprising:
triacetin as the sole polyol/polyol derivative
a mixture of 50% triacetin with 50% glycerol as the polyol/polyol derivative
a foil was manufactured from these mixtures and compared to a similar foil using glycerol as the sole polyol. It was found that partially or fully replacing glycerol with a polyol derivative (in this case triacetin) did not negatively impact flexibility of the resulting foil.

1. A nanocellulose-based composition for producing a foil or a coating of a substrate comprising based on dry weight:
15-40 parts nanocellulose;
15-50 parts polyol and/or a polyol derivative;
15-45 parts one or more additive selected from a cellulose ether, chitosan, chitin, casein, zein, globulin, albumin, prolamin, glutelin, gliadin, hordein, secalin, kafirin, and avenin or combination thereof;
wherein the sum of the parts adds up to one hundred parts, and
wherein the composition is a dry composition, suspension, emulsion or solution, and wherein the foil or coating for a substrate is compostable.

2. The nanocellulose-based composition according to claim 1, wherein the nanocellulose is selected from the group comprising microfibrillated cellulose, microcrystalline cellulose, cellulose nanocrystals, nanocrystalline cellulose, bacterial nanocellulose, plant derived nanocellulose, algae derived nanocellulose, fungi derived nanocellulose, and a combination of two or more thereof.

3. The nanocellulose-based composition according to claim 1, wherein the one or more additive is one or more selected from a cellulose ether and chitosan or combination thereof.

4. The nanocellulose-based composition according to claim 1, wherein the composition is a suspension or solution in a liquid, wherein the liquid comprises water, an alcohol, or a mixture thereof.

5. The nanocellulose-based composition according to claim 1, wherein the polyol is a low molecular weight polyol or a sugar alcohol.

6. The nanocellulose-based composition according to claim 1, wherein the one or more additive comprise one or more additive selected from HEC, MHEC and chitosan.

7. A method for preparing a nanocellulose-based intermediate product for producing a foil or a coating of a substrate, the method comprising mixing nanocellulose, polyol and/or a polyol derivative and one or more additives to obtain the nanocellulose-based composition as defined in claim 1.

8. The method according to claim 7, wherein the nanocellulose is pulverized prior to, during or after blending with the one or more additive.

9. The method according to claim 7, wherein, if the composition is a dry composition, the composition is suspended or dissolved in a liquid to obtain a nanocellulose-based suspension or solution.

10. The method according to claim 7, wherein the pH of the solution or suspension is adjusted to 5 to 8 pH by exposing the nanocellulose-based suspension or solution to a neutralization agent.

11. The method according to claim 7 wherein the method comprises the additional steps of:
applying the nanocellulose-based suspension or solution to a surface or a substrate, and
removing partly or completely the liquid from the nanocellulose-based suspension or solution, to obtain a nanocellulose-based foil or coating for a substrate.

12. Foil or coating for a substrate obtained or obtainable by the method of claim 11.

13. Foil or coating for a substrate according to claim 12, wherein the foil or coating for the substrate has
air permeability below 10 mL/min, as measured by the Bendtsen method using a pressure difference of 1.5 kPa; and/or
an oxygen transmission rate below 10 mL/(m²*day as measured at 23° C., <5% RH, atmospheric pressure, and/or
a grease/oil permeability (KIT) score of at least 10.

14. Use of the nanocellulose-based composition according to claim 1.

15. Use of the nanocellulose-based composition according to claim 1 as a coating for a substrate.

16. The nanocellulose-based composition according to claim 1, wherein the cellulose ether is an ionic cellulose ether or a non-ionic cellulose ether, or a combination thereof, more preferably wherein the cellulose ether is selected from the group comprising carboxymethyl cellulose (CMC), hydroxyethyl cellulose (HEC), methyl hydroxyethyl cellulose (MHEC), ethyl hydroxyethyl cellulose, methyl ethyl hydroxyethyl cellulose, hydroxypropyl methylcellulose, or a combination of two or more thereof, most preferably wherein the cellulose ether is HEC or MHEC or a combination thereof.

17. The nanocellulose-based composition according to claim 1, wherein the chitosan is selected from vegetal chitosan, fungal chitosan, synthesized chitosan, chitosan made water soluble or a combination of two or more thereof.

18. The nanocellulose-based composition according to claim 5, wherein the polyol is selected from glycerol, trimethylolpropane, pentaerythritol, polypropylene glycol, 1,4-butanediol, maltitol, mannitol, sorbitol, xylitol, erythritol, and isomalt; or wherein the polyol derivative is a polyol wherein one or more of the —OH groups have been replaced by a functional group,

19. The nanocellulose-based composition according to claim 18, wherein one or more —OH group has been replaced by an ester or an ether.

20. Foil or coating for a substrate according to claim 12, wherein the foil or coating for a substrate is impregnated with a substance, and/or wherein the foil is coated with a coating.
